(19) United States
(12) Patent Application Publication
(10) Pub. No.: US 2021/0240143 A1
(43) Pub. Date: Aug. 5, 2021

(54) CUSTOMIZABLE WORK STATIONS

(71) Applicant: Hewlett-Packard Development Company, L.P., Spring, TX (US)

(72) Inventors: Charles J. Stancil, Spring, TX (US); Richard E. Hodges, Spring, TX (US); Harold Merkel, Houston, TX (US); Ravi Shankar Subramaniam, Palo Alto, CA (US)

(73) Assignee: Hewlett-Packard Development Company, L.P., Spring, TX (US)

(21) Appl. No.: 17/054,535

(22) PCT Filed: Oct. 9, 2018
(86) PCT No.: PCT/US2018/054960

§ 371 Date: Nov. 11, 2020

(51) Int. Cl. G05B 13/02; G09G 5/10; G06K 9/00; G06T 7/70; G06T 7/20; F24F 11/64

(52) U.S. Cl. CPC G05B 13/028; G09G 5/10; G06K 9/00369; G06T 7/70; F24F 2120/12; F24F 11/64; G06T 2207/30201; G09G 2320/0626; G09G 2360/144; G06T 7/20

(57) ABSTRACT

A customizable work station is described with a computing device that has a local processor, a local display, a connector communicably coupled to the local processor, and a plurality of sensors that collect proximal information at the station to provide at least one personalized enhancement to a user of the station.

### BACKGROUND

When employees in workplaces use existing offices only occasionally or for short periods of time, offices are often left vacant. Shared work stations, sometimes referred to as hot desking, have emerged as an inexpensive solution in which multiple workers share a single physical work station or surface at different times. Companies worldwide are starting to provide shared office workspaces of various sizes and rental/lease periods. Not only does a shared workspace accommodate workers with flexible work schedules, it also enables workers traveling to various office sites to occupy vacant desks for a short period of time. It further enables permanent work stations that are dedicated to specific tasks to be shared by multiple employees. By sharing desks and/or offices, employees make more efficient use of company space and resources.

### DESCRIPTION OF THE DRAWINGS

FIG. 1a is a block diagram of a customizable work station that provides at least one personalized enhancement according to an example of principles described herein.

FIG. 1b is a top view of an example workplace according to an example of principles described herein.

FIG. 2 is a perspective view of a customizable work station with personalized enhancements, according to an example of principles described herein.

FIG. 3 is a perspective view of example personalized enhancements within a customizable work station, according to an example of the principles described herein.

FIG. 4 is a diagram of an example computing device for personalizing a customizable work station, according to an example of the principles described herein.

FIG. 5 is a flowchart of a method for providing a personalized enhancement, according to an example of principles described herein.

FIG. 6 is a flowchart of a method for providing a personalized enhancement, according to another example of principles described herein.

FIG. 7 is a flowchart of a method for providing a passive or personalized enhancement, according to another example of principles described herein.

FIG. 8 is a diagram of a computer program product according to another example of principles described herein.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

A basic customizable work station refers to a work space used by many individuals and may include little more than a work surface, a chair, and a monitor. While providing convenient access, the customizable work station often still seems foreign and uncomfortable to a user with little to no comforts of a familiar space. Strides have been made to provide customized settings for features such as desk height, temperature, lighting, presence detection, etc., however, a loss in privacy and efficiency is caused by sharing this information over a server.

To this end, a customizable work station of the present specification provides personalized enhancements that may be set at a local level. For example, the user goes to a customizable work station in a work place, connects their computing device to the customizable work station, and is instantly greeted by name via the display or audio output. The sit down/stand up desk automatically rises to a preferred ergonomic height for the user. Overhead lights brighten or dim depending on the time of day and nearby ambient lighting. Filters mute out nearby conversations providing a quiet space for needed concentration. Temperature, humidity, and air flow automatically adjust to default normal working conditions or pre-defined settings. The user begins to perform in an optimal environment, almost as if they never left their home computer. Additionally, the user experiences privacy, with their personal information and settings remaining at the customizable work station.

The customizable work station described includes a station with a local processor and a local display output. A connector is communicably coupled to the local processor. When a computing device is connected to the connector, the local processor begins to collect proximal information at the station. The local processor uses the information to provide at least one personalized enhancement for a user. When the local processor does not detect a computing device or a user, the display outputs passive information.

A method is also provided herein for controlling a customizable work station. The method includes detecting a computing device connected to a connector of a local processor. The method further includes the use of at least one sensor to provide proximal information to the local processor. The processing of the local processor is isolated from a central server. Upon receiving proximal information from the at least one sensor, the local processor provides at least one personalized enhancement to a feature associated with the customizable work station.

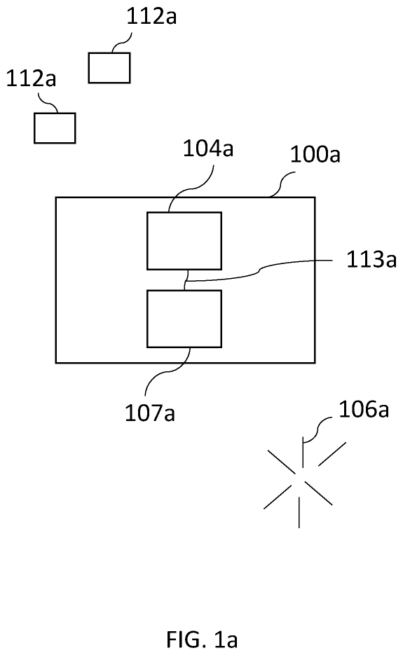

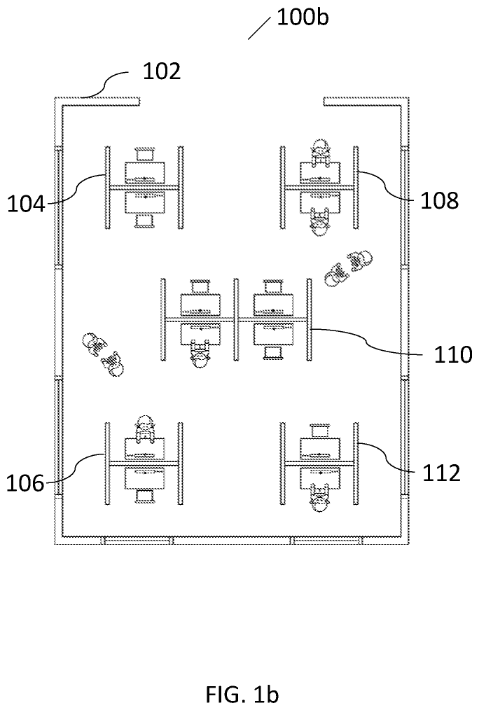

Turning to FIG. 1a, a block diagram illustrates a work station 100a that provides at least one personalized enhancement 106a. The work station 100a includes a local processor 104a that is communicably coupled to a connector 113a which is communicably connected to a computing device 107a. The computing device may be portable or stationary. When the computing device 107a is connected to the connector 113a, the local processor 104a begins to collect proximal information at the station through at least one sensor 112a. The local processor 104a uses the proximal information to provide at least one personalized enhancement 106a for a user.

Turning to FIG. 1b, a top view of an example workspace 100b is depicted. Within the workspace 100b is a working environment 102 that includes stations 104, 106, 108, 110, and 112. Stations 104, 106, 108, and 112 each have two desks facing each other and are separated by walls that partially surround each desk area. Station 110 includes four desks which are partitioned off from each other by walls. The workspace includes gathering areas where people gather and converse. While specific reference is made to a workspace 100b with a particular setup, other types of workspaces may be implemented as well. Other types of workspaces include closed offices with doors, tables or other work surfaces without partitions or walls, and other types of workspaces. Within each desk area, personalized enhancements are provided so as to customize a work environment that is specific to each user individually for the time allotted to that user.

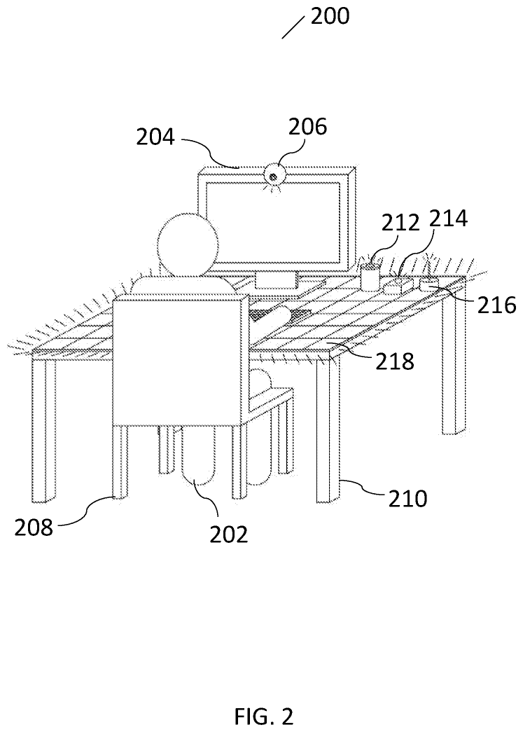

Turning to FIG. 2, an example work station 200 is shown that includes a user 202 sitting at a table 210 and a display 204. A variety of sensors are present at the work station 200 to capture proximal information and send it to a local processor (not shown). Example sensors include a camera 206, audio sensor 212, humidity sensor 214, light/temperature sensor 216, pressure sensor 218, as well as other sensors not shown, including but not limited to, weight sensors, radio-frequency identification (RFID) sensors, presence sensors, biometric readers, microphones, gesture sensing devices, tactile/touch sensors (e.g., associated with an electronic display screen or emissive surface, etc.).

The sensor devices are provided to sense activities and conditions within the work space. Activation of the sensors may be based on detecting proximal information, such as a presence of a user, a connection of a computing device to the local processor, detection of pressure of a user's hands on a work surface, detection of pupil movement of eyes or limbs, detection of temperature fluctuations of the workspace environment, or by another activation trigger. Activated sensors may also detect this type of information. The proximal information gathered is sent to a local processor that is local to the station or to a plurality of nearby stations. Note that the local processor may further receive control commands from the user.

Variations further include a plurality of stations that are locally related. For example, two workstations may have a common local processor. This setup allows a large group needing two workstations to share the same personalized enhancements at the same time or at different times in an efficient manner.

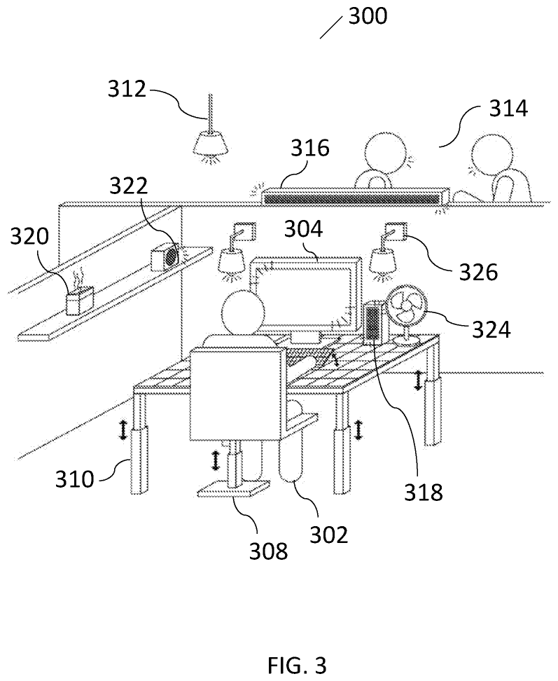

Turning to FIG. 3, another example work station 300 in a workspace environment is shown. The work station 300 depicts various actuations that may be executed to provide personalized enhancements based on information that is received from the various sensors. For example, a display 304 may be raised to eye level or to another desired level and/or tilted to avoid straining neck muscles. A chair 308 or other seating surface and/or a work surface or table 310 may be raised or lowered to accommodate the height of a user 302. The table and/or chair height may also be changed periodically to help the user 302 stay active while working. Surrounding air flow 324 and temperature 322 may be automatically maintained at optimal levels according to standards or according to levels set by the user 302. Interior/display lighting 326 may be adjusted for optimal vision and concentration.

Sensors may gather not only proximal information, but also external information, or in other words, information that originates from outside the work station 300. For example, ambient conversation and noise 314 may be detected by sensors and in response, ambient control 316 is used to filter the noise. In another example, at least one sensor gathers information about external lighting 312 (e.g., external ceiling lights, lamps, window light, adjacent station lighting, etc.) to actuate proximal station lighting for brightening or dimming interior lighting.

Sensors and actuators may work in a variety of combinations. For example, information from one sensor may result in one actuated personalized enhancement. Information from one sensor may result in two or more actuated personalized enhancements. Information from two or more sensors may result in one actuated personalized enhancement. And, information from two or more sensors may result in two or more actuated personalized enhancements.

An example of one sensor resulting in one actuated personalized enhancement includes a camera 206 sensing a user's height and adjusting the height of the user's table 310 as the actuated personalized enhancement. Another example is a temperature sensor 216 sensing the proximal temperature and then adjusting the temperature 322 to be in accordance with a preferred temperature.

An example of one sensor resulting in two or more actuated personalized enhancements includes a camera 206 sensing the user's height with the personalized enhancement being an adjustment to the height of the user's chair 308 and the table 310. In another example, a light/temperature sensor 216 senses window lighting and the actuation dims interior overhead lighting as well as display lighting 326.

An example of two or more sensors resulting in one actuated personalized enhancement includes a camera sensing a head tilting lower than normal along with a pressure sensor sensing that hands are applying greater than normal pressure on a work surface. The local processor 105a determines that the droopy head and increased pressure are indicative of the user falling asleep and the resultant actuated personalized enhancement is that the display 304 output nudges the user to wake up. This may include an audible alert or visual cue, for example. In another example, the ambient noise sensor 212 senses heightened decibel levels of conversation in the background and the ambient temperature sensor 216 senses a heightened temperature. Information from the two sensors 212 and 216 is used in combination by the processor to determine that an abnormal condition in the building may be present and an alert is sent to the user with notification that there may be a serious situation at hand.

An example of multiple sensors resulting in multiple actuated personalized enhancements includes a camera 206 sensing a body angle over a period of time and a pressure sensor 218 sensing pressure of the body on the user's table. The response may be that the table height 210, chair 208 and display 204 are all adjusted to various levels to ensure that the user changes position and has various activity while working.

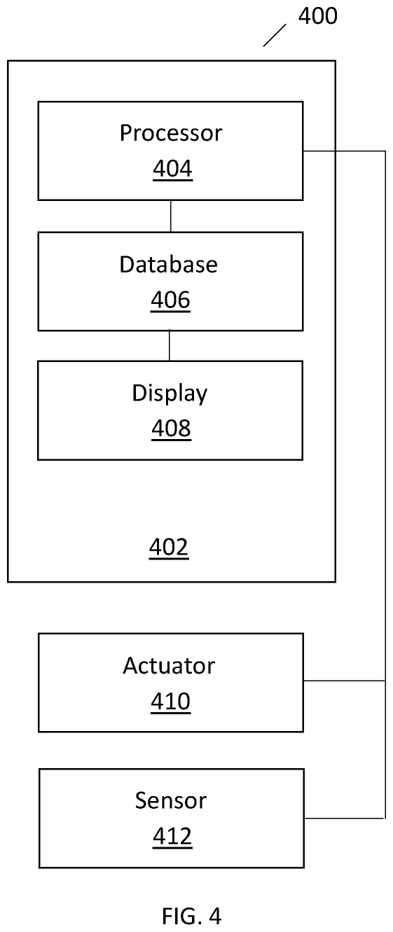

An example sensor-rich workspace that enables an office as a service includes a system 400 in FIG. 4, which is represented according to an example of principles described herein.

The system 400 includes a local computing device 402 that is isolated from a central server. While there may be some communications or connectivity with a central or outside server, the processing for the personalized enhancements at the customizable work station may be kept isolated. The computing device 402 includes a local processor 404, a database 406, and a display output 408. The system 400 implements the workings of the local processor 404 using an actuator 410 and a sensor 412.

The display output 408 displays information to a user. Such information may include communications that are based on information that is received by the local processor 404. The information is processed to perform actuation of displaying communications on a display. Alternatively, the information may be gathered from a local database 406 that collects and stores each user's information. Also, displayed information may be passive information that is not intended for a specific user. This may include, for example, displaying a particular customizable work station reference number or a map of the area around the customizable work station highlighting meeting spaces, breakrooms, and restrooms. The display output 408 may further display emergency notifications or advertisements.

Passive information that is displayed on a display may be stored in the database 406, be updated periodically, or have real-time capabilities over the network. Passive information may include, for example, information related to a mapping between a particular user and certain settings or a mapping between sensor output and adjustments, etc. The local processor 404 may be connected over the network but may switch to an isolated state from the server when user presence is detected or a computing device is connected to the local processor 404. Alternatively, there may be more than one local processor 404 such that remote communications are handled separately from the local processor 404 handling local sensors and actuation.

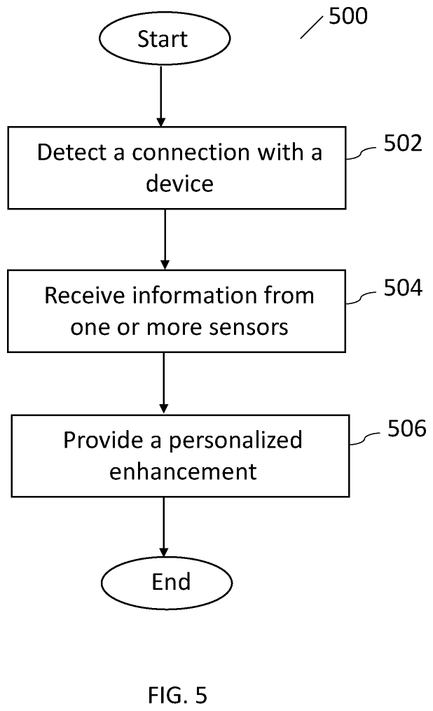

FIG. 5 provides a flow chart of an example method 500 for providing a personalized enhancement. The method 500 in FIG. 5 starts when detection of a device (block 502) being connected to a local processor (404, FIG. 4) is made. The connection may be a physical connection to a port of the local processor 104a (404, FIG. 4). Alternatively, the connection may be a wireless connection. The connection establishes a presence of a user at a customizable work station 200. At least one sensor 412 within the workspace 100b begin to sense information and provide that information to the local processor (block 504). Sensors used include a camera 206, audio sensor 212, humidity sensor 214, temperature sensor 216, pressure sensor 218, as well as other sensors not shown, including but not limited to, weight sensors, RFID sensors, presences sensors, biometric readers, microphones, gestures sensing devices, tactile/touch sensors (e.g., associated with an electronic display screen or emissive surface), etc., as described previously with reference to FIG. 2.

Types of information collected by the sensors include proximal information that is information within the immediate workspace environment. The information may also include information from outside of the workspace. In either case, the information is processed by the local processor (404, FIG. 4) to be stored in a local database 406 (FIG. 4) and may include, for example, user presence, ID badge information, facial structure, posture, breathing status, neck angle, head position and movement, appendage position and movement, height, eye blinking rate, etc. The information may be used to perform data analytics at the local level exclusively or that are sent to an outside server.

Based on the determinations made with the supplied information, at least one personalized enhancement is provided (block 506) which may be executed by actuators 410. The actuation may affect various structures and devices, as shown in FIG. 3 (304, 308, 310,312, etc.). Such actuation may further be time dependent. For example, the air conditioning 322 may be actuated during the day but not during the night.

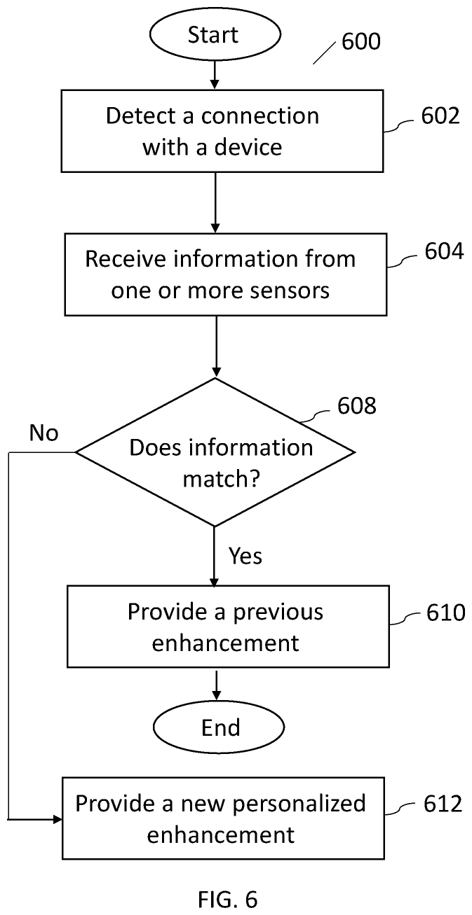

FIG. 6 illustrates a method 600 in which current information is compared with previous information to provide a personalized enhancement. Like method 500, method 600 begins when a connection 113a to the local processor 104a is detected (block 602). Information is received by at least one sensor (block 604) and supplied to the local processor (404, FIG. 4). The information from the current connection is compared (block 608) with previous information from past use with other connections. The previous information from past use may include sensed information, determinations made with that information, data analytics associated with that information, and personalized enhancements that were previously performed based on that information.

If a match with previous information is made (block 608, determination YES), previous enhancements may be implemented (block 610) in accordance with the determinations and analytics made previously. Thus, proximal information of a previous user from the database is used to provide personalized enhancements associated with the previous user when there is a match between the proximal information of a previous user and the proximal information of a current user.

In one example, a user may step away from the desk 310 to use the restroom. The local processor 404 does not receive information for a few minutes and goes into standby mode in which passive information is displayed on the monitor 304 (FIG. 3). The sensors 412 in the standby mode are not actively engaged in sensing information, rather, they are periodically sensing information. When the user returns and the camera 206 senses information during its periodic rotation, the camera 206 retrieves the user's height, appendage movement, and other biometric related data that enables the local processor to make a comparison to previous information and thereby confirm that the user is the same user that was previously in the workspace. Based on this determination, the local processor immediately resumes providing the same enhancements 106a that were previously associated with the user.

By doing a local comparison of current information with previous information, acts of scanning or otherwise recognizing identification tags (e.g., RFID tags, badges, etc.) that would normally be done over a server, are avoided. That is, there is no need to send personal or confidential information associated with an identification tag over a network, which transmission could be susceptible to hacking. In other words, with sensors acting at the local level and the processer providing personalized enhancements at the local level, user confidentiality is preserved because information is processed and then stored at a local level. With less information being transferred over the network, less power and energy is needed by the network which ensures a more efficient handling of data.

If the current information does not match the previous information (block 608, determination NO), a new personalized enhancement 106a based on the current information is provided (block 612). Thus, proximal information of a previous user from the database is not used to provide personalized enhancements associated with the previous user when there is no match between the proximal information of a previous user and the proximal information of a current user.

In some examples, the personalized enhancements 106a that are applied may be user-defined. That is, although personalized enhancements may be determined by the local processor 404, the user has the option to define preferences that override default enhancements so that the determinations made by the local processor do not wind up providing undesired enhancements. For example, a user may set a desirable temperature and that temperature will be saved as a personalized enhancement that is associated with the user and will not be overridden by a determined optimal temperature or a standardized temperature.

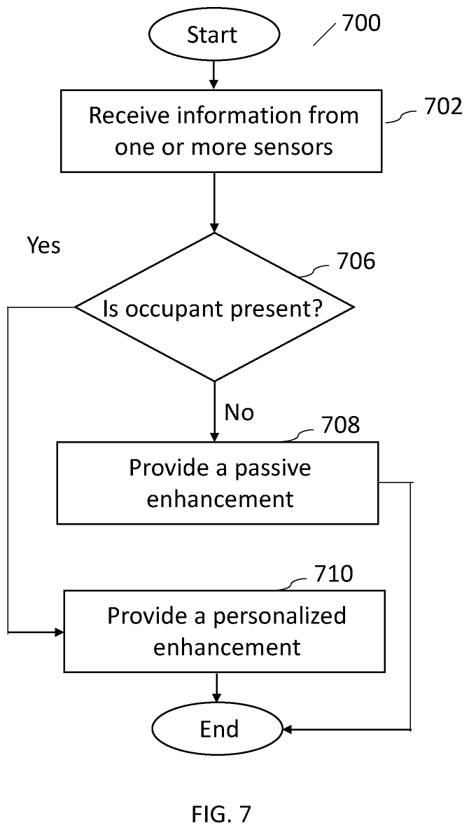

As stated previously, although a connection may activate the sensors 412, some sensors may periodically be activated, regardless of a connection to a local processor. For example, at least one sensor may be on at a constant or semi-constant state without any connectivity present. FIG. 7 illustrates a method 700 in which information is received (block 702) from at least one sensor in this fashion. That is, the information may be received while there is connectivity, or alternatively, when there is no connectivity. If the occupant is present (block 706), a personalized enhancement may be provided (block 710). If no occupant is present (block 706), a passive enhancement is provided (block 708). For example, a camera that detects no presence of a user may continue to display a reference number assigned to the workstation 200.

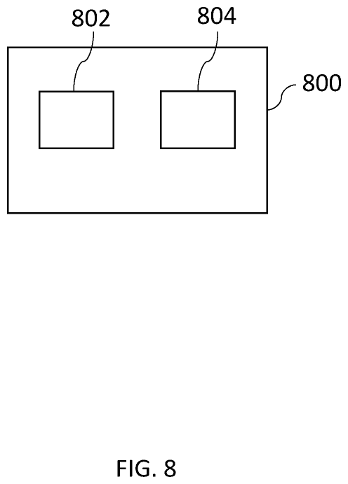

Referring now to FIG. 8, a computer program product 800 is anticipated that is associated with principles herein. Particularly, a computer program product 800 is anticipated that comprises at least one computer-readable storage media 802 that include computer executable instructions. The instructions are structured such that, when interpreted by a local processor 804 associated with the computer program product, cause the computer program product 800 to perform the method steps described in reference to the previous methods described herein.

What is claimed is:

1. A customizable work station comprising:
a computing device that comprises a local processor and a local display output;
a connector communicably coupled to the local processor, the connector to connect with a computing device; and
a plurality of sensors connected to the local processor, the plurality of sensors to collect proximal information at the station and provide the information to the local processor in response to the computing device being detected by the local processor,
the local processor to use the information to provide at least one personalized enhancement for a user of the station, the at least one personalized enhancement further being stored by the local processor.

2. The customizable work station of claim 1, further comprising at least one actuator controlled by the local processor, the at least one actuator actuating at least one structural component of the work station to provide the at least one personalized enhancement, the structural component to affect at least one of the following:
environmental lighting,
display lighting,
air flow,
humidity,
external communication,
internal communication,
audio levels,
ambient noise suppression,
desk ergonomics,
chair ergonomics, and
display ergonomics.

3. The customizable work station of claim 1, wherein at least one sensor of the plurality of sensors comprises a camera to provide to the local processor with image capture information associated with at least one of:
user presence,
ID badge,
facial structure,
posture,
breathing,
neck angle,
head position and movement,
body position and movement,
appendage position and movement,
height, and
eye movement and blinking rate.

4. The customizable work station of claim 1, further comprising
a machine-learning database comprising previously gathered proximal information and personalized enhancements associated with a previous user;
the local processor to compare the previously gathered proximal information of the previous user from the database with current proximal information from the plurality of sensors to determine whether a current user has previously used the station; and
the local processor to provide personalized enhancements associated with the previous user to the current user when the current user information matches the previously gathered proximal information.

5. The customizable work station of claim 1, in response to at least one sensor of the plurality of sensors detecting an absence of a user at the station, the local processor to display passive information on the local display output.

6. The customizable work station of claim 1, wherein at least one sensor of the plurality of sensors determines temperature and the at least one personalized enhancement comprises making an adjustment to an air conditioning unit.

7. The customizable work station of claim 1, wherein the proximal information is processed by at least one of the following configurations:
one sensor resulting in one personalized enhancement,
one sensor resulting in two or more personalized enhancements,
two or more sensors resulting in one personalized enhancement, and
two or more sensors resulting in two or more personalized enhancements.

8. The customizable work station of claim 1, wherein the at least one personalized enhancement is time dependent.

9. The customizable work station of claim 1, wherein at least one sensor of the plurality of sensors gathers ambient light intensity information and the at least one personalized enhancement comprises at least one of dimming the local display output and dimming a light at the work station.

10. The customizable work station of claim 1, wherein at least one sensor of the plurality of sensors detects pressure applied on a desk top to determine the at least one personalized enhancement.

11. A method of providing a customizable work station comprising:
detecting a computing device connected to a connector of a local processor, wherein processing by the local processor is isolated from a central server;
receiving proximal information from at least one sensor that is communicably connected to the local processor; and
providing, based on received proximal information, at least one personalized enhancement to a feature associated with a customizable work station.

12. The method of claim 11, further comprising actuating at least one actuator to provide the at least one personalized enhancement, the structural component to affect at least one of the following:
environmental lighting,
display lighting,
air flow,
humidity,
external communication,
internal communication,
audio levels,
ambient noise suppression,
desk ergonomics,
chair ergonomics, and
display ergonomics.

13. The method of claim 11, further comprising
storing the at least one personalized enhancement in a machine-learning database;
comparing previously gathered proximal information from the database with current proximal information to identify whether or not a current user is a previous user of the customizable work station; and
providing at least one previously stored personalized enhancements associated with the previous user to the current user when the current proximal information matches the previously gathered proximal information.

14. The method of claim 11, further comprising providing a bimodal display functionality by:
detecting a state of no occupancy, and
presenting passive information on a display of the work station.

15. A computer program product comprising at least one computer-readable storage media having thereon computer-executable instructions that are structured such that, when interpreted by a local processor associated with the computer program product, cause the computer program product to:
detect a computing device connected to a connector of the local processor, wherein processing by the local processor is isolated from a central server;
receive proximal information from at least one sensor that is communicably connected to the local processor; and
provide, based on received proximal information, at least one personalized enhancement to a feature associated with a customizable work station.
